(19) United States
(12) Patent Application Publication
(10) Pub. No.: US 2019/0018017 A1
(43) Pub. Date: Jan. 17, 2019

(54) Integrated Analysis To Determine Prognosis After Treatment For Primary Breast Cancer

(71) Applicants: NantOmics, LLC, Culver City, CA (US); Nant Holdings IP, LLC, Culver City, CA (US)

(72) Inventors: Stephen Charles Benz, Santa Cruz, CA (US); Todd Hembrough, Culver City, CA (US); Shahrooz Rabizadeh, Los Angeles, CA (US); John Zachary Sanborn, Santa Cruz, CA (US); Charles Joseph Vaske, Santa Cruz, CA (US); Fabiola Cecchi, Culver City, CA (US); Peter Fasching, Culver City, CA (US); Patrick Soon-Shiong, Los Angeles, CA (US)

(73) Assignees: NantOmics, LLC, Culver City, CA (US); Nant Holdings IP, LLC, Culver City, CA (US)

(21) Appl. No.: 16/060,638

(22) PCT Filed: Dec. 11, 2016
(86) PCT No.: PCT/US16/66048

§ 371 Date: Jun. 8, 2018

Related U.S. Application Data
(60) Provisional application No. 62/265,928, filed on Dec. 10, 2015.

(51) Int. Cl. G01N 33/574; A61K 39/395; C07K 16/32; A61K 31/337

(52) U.S. Cl. CPC G01N 33/57484; G01N 33/57415; A61K 31/513; C07K 16/32; A61K 31/337; A61K 39/39558

(57) ABSTRACT

Various protein markers can be used as post-treatment relapse predictors in HER2 positive breast cancer. Notably, these markers appear to be independent of the size of the tumor, metastasis status, grade, and hormone receptor status. In addition, HER2 quantities were in large part not correlated with likelihood of relapse.

This application claims the benefit of priority to U.S. provisional application having Ser. No. 62/265,928, filed on Dec. 10, 2015.

### FIELD OF THE INVENTION

The field of the invention is omics analysis, and especially as it relates to panomics analysis for breast cancer.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Breast cancer is a complex disease in which tumors exhibit a large biologic diversity and spectrum of clinical behaviors. As a consequence, different tumors will have significantly different responses to the same therapy. Breast cancer is often classified based on various molecular markers, and at least five subtypes of breast cancer are known: two luminal subsets within estrogen receptor (ER)-expressing tumors, and three subsets in mostly ER-negative tumors (HER2, normal breast-like, and the basal-like subtypes). Unfortunately, classification of breast cancer will not necessarily simplify the choice of proper treatment, nor help assist in accurate prediction of treatment outcome.

Among other examples, the ‘triple-negative’ (TN) (ER, progesterone receptor (PR), and HER2) class of breast cancer has often poor prognosis that is compounded by a lack of established therapies that target this subtype of breast cancer. Similarly, HER2-positive breast cancer (human epidermal growth factor receptor 2 expressing breast cancer) is often difficult to treat due to the relatively fast growth, propensity to metastasize, and high recurrence rate. Indeed, despite improvements in the treatment of HER2-positive breast cancer, recurrence is a persistent problem, possibly based on acquired resistance to HER2-targeted agents. A number of mechanisms of resistance have been proposed, including: Mutations in PIK3CA, lack of antibody-dependent cellular cytotoxicity, and low expression levels of HER2. Still further, predictability of treatment outcome for HER2-positive breast cancer is confounded by the large diversity of primary tumor size, lymph node involvement, stages, and grade.

Under current therapies, HER2 positive breast cancer is often treated using an anti-HER2 antibody and at least an anthracycline (e.g., epirubicin, doxorubicin, etc.) and a taxane (e.g., docetaxel, paclitaxel, etc.). For example, current therapies often employ after primary surgery a number of treatment cycles of FEC (5-fluorouracil, epirubicin, cyclophosphamide) followed by a number of treatment cycles of docetaxel or docetaxel plus gemcitabine, or use an adjuvant chemotherapy that includes an anthracycline and a taxane. In addition to these drugs, an anti-HER2 antibody (e.g., trastuzumab) is typically given to the patient for a total of 12 months. Despite such treatment regimens, relapse will occur in a significant fraction of patients, and there is currently no known method for predicting treatment outcome for HER2-positive breast cancer.

Therefore, there is a continuing need for systems and methods of predicting treatment outcome for HER2-positive cancer, especially where treatment uses an anti-HER2 antibody and at least an anthracycline and a taxane.

### SUMMARY OF THE INVENTION

The inventive subject matter is drawn to various compositions, systems, and methods of predicting treatment outcome for HER2-positive cancer, especially where treatment uses an anti-HER2 antibody and at least an anthracycline and a taxane. In most typical aspects of the inventive subject matter, suitable predictive markers of treatment success include TLE3 (transducin-like enhancer protein 3), XRCC1 (X-ray repair cross-complementing protein 1), RRM1 (ribonucleotide reductase catalytic subunit M1), and/or MGMT (O(6)-methylguanine-DNA-methyltransferase).

In one aspect of the inventive subject matter, the inventors contemplate a method of predicting post-treatment relapse in a patient treated for a HER2-positive breast cancer. Most typically the patient treatment comprises administration of an anti-HER2 antibody (e.g., herceptin) and at least an anthracycline (e.g., epirubicin, doxorubicin, etc.) and a taxane (e.g., docetaxel, paclitaxel, etc.). In one step of such methods, a breast cancer sample is obtained from the patient, and presence and/or quantity of a at least one marker is determined, wherein the marker is TLE3, XRCC1, RRM1, or MGMT. In still another step, the presence and/or quantity of the marker are then used to predict a likelihood of post-treatment relapse in the patient. Presence or higher than normal quantities (as compared to same patient non-cancer tissue) of these markers are associated with a lower likelihood of relapse within 5 years.

While in some aspects the patient treatment comprises three administration cycles of FEC (5-fluorouracil (5FU), epirubicin, and cyclophosphamide) followed by three cycles of docetaxel or docetaxel plus gemcitabine, treatment in other aspects may comprise an adjuvant chemotherapy with an anthracycline and a taxane. Moreover, it is generally contemplated that administration of the anti-HER2 antibody is performed over an extended period of time (e.g., 12 months).

It is still further contemplated that the step of determining the presence and/or the quantity of the marker is performed using at least one, or at least two, or each of DNA omics analysis (e.g., whole genome or exome analysis), RNA omics analysis (e.g., RNAseq), and proteomics analysis (e.g., selective reaction monitoring mass spectroscopy). Therefore, and viewed from a different perspective, determination of the presence and/or quantity of the marker may include a determination of a gene copy number, a gene expression level, and/or protein level.

With respect to the prediction of the likelihood of post-treatment relapse in the patient, it is contemplated that the prediction is independent of the size of the primary tumor, the lymph node status, the grade, and the hormone receptor status. In addition, it is also contemplated that the prediction of the likelihood of post-treatment relapse in the patient is also not correlated with a HER2 quantity in the breast cancer sample. Most typically, presence, increased copy number, or increased presence of the marker will be predictive of a lower likelihood of post-treatment relapse.

Consequently, the inventors also contemplate the use of the presence and/or quantity of at least one of TLE3, XRCC1, RRM1, and MGMT in the prediction of a treatment outcome of a HER2-positive breast cancer, wherein the treatment comprises administration of an anti-HER2 antibody and at least an anthracycline and a taxane.

Suitable treatments in such use may include three administration cycles of FEC (5-fluorouracil (5FU), epirubicin, and cyclophosphamide) and three administration cycles of docetaxel or docetaxel plus gemcitabine, or adjuvant chemotherapy with an anthracycline and a taxane. In addition, the treatment will also typically include administration of an anti-HER2 antibody is performed over an extended period (e.g., 12 months).

Presence and/or quantity in contemplated uses are typically determined using at least one, at least two, or each of an DNA omics analysis, an RNA omics analysis, and a proteomics analysis. Such analysis may be performed in various manners, however, it is typically preferred that the analysis includes measuring at least one of a gene copy number, a gene expression level, and a protein level. As noted above, the prediction of the treatment outcome is typically independent of the size of the primary tumor, the lymph node status, the grade, and the hormone receptor status, and is further independent on the quantity of HER2 in the tumor.

Various objects, features, aspects and advantages of the inventive subject matter will become more apparent from the following detailed description of preferred embodiments, along with the accompanying figures in which like numerals represent like components.

### BRIEF DESCRIPTION OF THE DRAWING

FIG. 1 is a schematic flow chart illustrating selection of patients for an exemplary study according to the inventive subject matter.

FIG. 2 is a table showing parameters of the patients selected from the flow chart of FIG. 1.

FIG. 3 is a table listing selected proteins identified by proteomics analysis that are associated with positive treatment outcome in a statistically significant manner.

FIG. 4 is a graph exemplarily depicting a lack of an overall correlation of HER2 protein levels with treatment outcome.

FIG. 5 is a graph exemplarily depicting correlation (by quintiles) of TLE3 protein levels with treatment outcome.

FIG. 6 is an exemplary graphical representation of selected mutations in a HER2 positive tumor relative to normal tissue of the same patient.

FIG. 7 is a graph exemplarily depicting correlation between DNA and protein, RNA and protein, and DNA and RNA for selected genes.

FIG. 8 is a graph comparing selected parameters for patient samples in the present inventive subject matter versus corresponding TCGA data.

### DETAILED DESCRIPTION

The inventors have now discovered specific markers that are highly accurate for the prediction of treatment outcome of specific HER2 breast cancer treatments. Advantageously, predictions using these markers are independent of the size of the primary tumors, the lymph node status, the tumor grade, and the hormone receptor status. As is discussed in more detail below, the markers presented herein are especially suitable for the prediction of treatment outcome where the patient is treated with an anti-HER2 antibody and at least an anthracycline and a taxane. Since HER2 tumors exhibit substantial diversity with respect to biological and behavioral parameters, the inventors used a panomic approach to ascertain that DNA markers identified with genomics were also relevant with respect to their transcription and translation into the corresponding proteins. Thus, and viewed from a different perspective, the inventive subject matter is also directed to a comprehensive panomics approach that integrates whole genome sequencing (WGS), RNA sequencing (RNAseq) and quantitative proteomics (SRM-MS) to determine associations between tumor molecular profiles and prognosis/therapeutic outcome among patients with HER2-positive breast cancer.

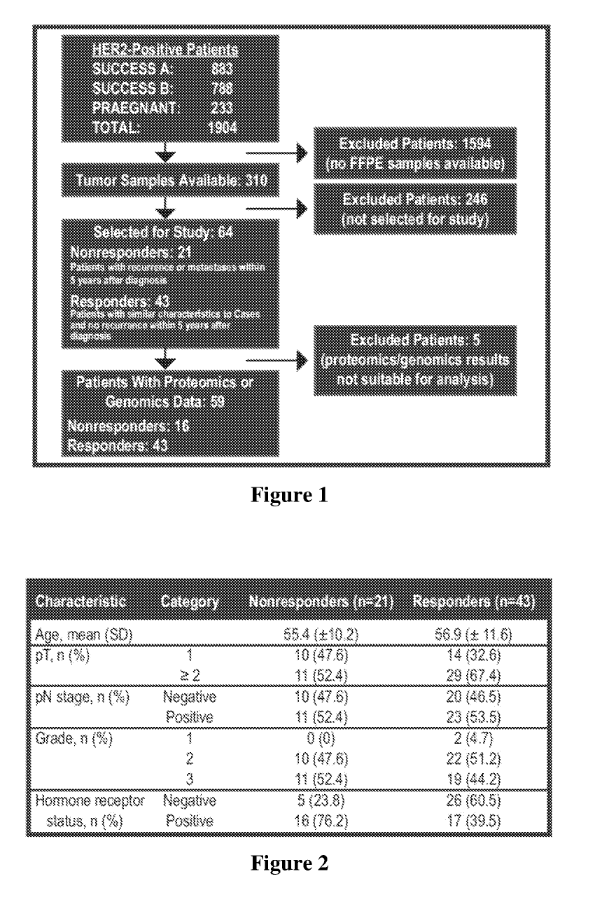

More specifically, as schematically shown in FIG. 1, the inventors enrolled patients from the SUCCESS A, SUCCESS B, and PRAEGNANT studies for which various data were available. SUCCESS A and SUCCESS B studies included HER2 positive high-risk breast cancer patients after primary surgery. Here, all HER2-positive patients received a standard chemotherapy, including three cycles of FEC (5-FU, epirubicin, and cyclophosphamide) that was followed by three cycles of docetaxel or docetaxel plus gemcitabine. The anti-HER2 antibody trastuzumab was given to all patients for a total of 12 months. PRAEGNANT is a registry of metastatic breast cancer patients. All patients selected from this study received a standard adjuvant chemotherapy, including anthracyclines and taxanes. Trastuzumab, an anti-HER2 antibody, was given to all patients for a total of 12 months.

Of a total of 1904 patients, 1594 patients were excluded from the analysis due to lack of formalin fixed paraffin embedded samples that would otherwise be used for proteomics analysis. Of the remaining 310 patients, a further 246 were not selected for this study. This left 64 patients for analysis in which 21 patients were non-responders (i.e., experienced recurrence or metastases within 5 years after treatment) and in which 43 patients were responders (i.e., no recurrence or metastases within 5 years after treatment). Another five patients were excluded for lack of suitable genomics and/or proteomics data. Therefore, the final study population was 59 patients, with 16 non-responders and 43 responders.

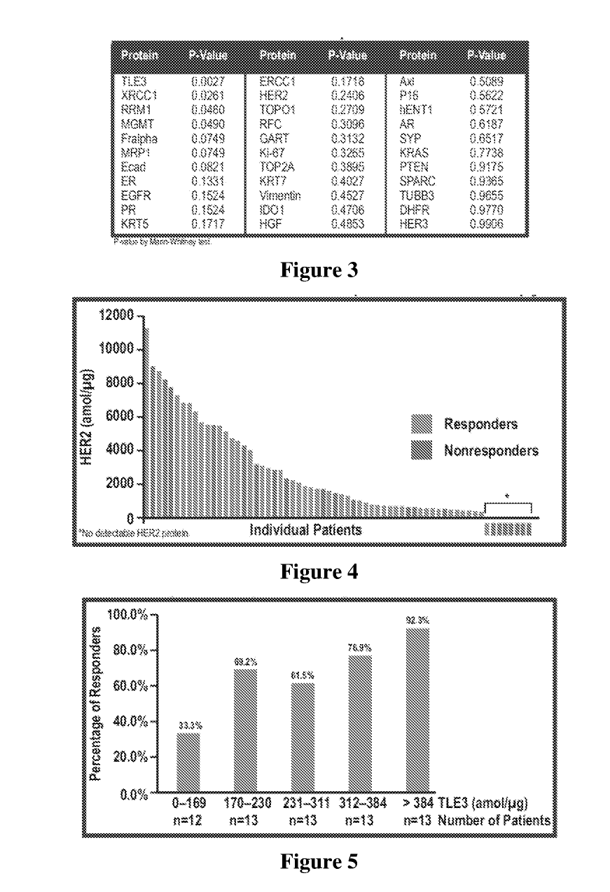

FIG. 2 provides selected patient criteria. Most notably, the patient pool included patients with relatively small primary tumors (T1) as well as patients with larger tumors (≥T2). Additionally, the patients included in the study had different stages of lymph node involvement (positive, negative) and also fell into different grades (between 1-3, inclusive). Moreover, the patient population was also mixed with respect to hormone receptor status (i.e., estrogen receptor, progesterone receptor). Such diverse patient population would ordinarily not be expected to provide a single marker with statistically significant prediction power. Unexpectedly, the inventors discovered after panomic analysis using DNA (including mutational analysis, and copy number analysis), RNA (using quantitative RNA analysis and RNA sequence analysis), and protein data (using SRM-MS from FFPE tissue sections) that various markers positively correlated with positive treatment outcome (i.e., responder status) at notably high statistical significance having a p-value of equal or less than 0.050. FIG. 3 depicts a collection of exemplary proteins tested and their statistical significance associated with responder status. As can be readily taken from the Table in FIG. 3, the most relevant markers associated with responder status in patients treated with an anti-HER2 antibody and at least an anthracycline and a taxane as noted above were TLE3 (transducin-like enhancer protein 3), XRCC1 (X-ray repair cross-complementing protein 1), RRM1 (ribonucleotide reductase catalytic subunit M1), and MGMT (O(6)-methylguanine-DNA-methyltransfer-ase). While most of these proteins have already been described in at least some capacity as cancer markers or cancer associated proteins, they were heretofore not known as predictive markers for treatment outcome for HER2 positive breast cancer in patients treated with an anti-HER2 antibody and at least an anthracycline and a taxane as noted above.

Moreover, using protein analysis from FFPE sections of tumors it was also observed that the amount of HER2 expression in the tumors did not (to a very large degree) correlate with responder status as can be taken from FIG. 4. Here, HER2 protein concentration as measured in amol/μg total protein varied between lower detection limit about 11,000 amol/μg and responders and non-responders were substantially randomly distributed among varying quantities of HER2 protein. Only samples with HER2 at or below lower detection limit were associated with non-responder status (which is arguably to be expected where the treatment is based in part on anti-HER2 antibodies), and patients with low levels (i.e., bottom quintile) of detectable HER2 protein had a notably worse response rate (41.7%) than those with higher HER2 expression (73.1%). Thus, when considering the entire patient population pool, HER2 expression status was not significantly associated with prediction of recurrence. Such result is especially unexpected as treatment of the patients had an anti-HER2 antibody as modality, which would ordinarily be expected as a predictive marker.

With respect to protein quantities of the markers and strength of response prediction, the inventors further noted that for TLE3 the strength of protein expression in the FFPE samples did even stronger correlate where more TLE3 was present. FIG. 5 exemplarily shows responders to treatment as a function of quintiles for TLE3 expression as measured by SRM-MS from FFPE samples. Here, the quintile for highest expression (>384 amol/μg) had the highest percentage of responders (92.3%), with declining percentages at lower expression levels. Therefore, the prediction of the likelihood of post-treatment relapse in the patient may not only be based on a quantitative result (e.g., expressed vs. not expressed, or expressed at a higher level than matched normal control), but also include a quantitative result with respect to the marker.

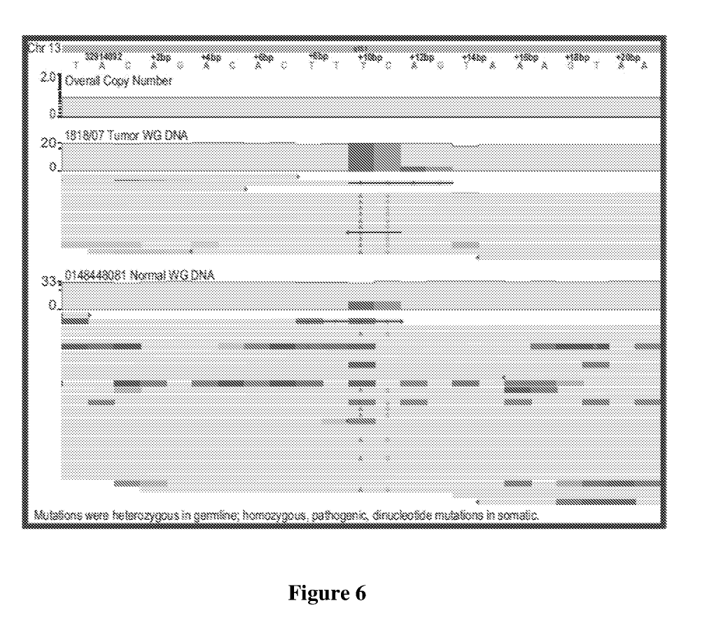

Similarly, at least some genes also appeared to be correlated with response status, and particularly BRCA2 as is exemplarily illustrated in FIG. 6. Here, a customized genome browser showing tumor whole genome DNA versus matched normal genome DNA identified a LOH (loss of heterozygosity)-mediated selection of a pathogenic dinucleotide BRCA2 variant as a potential driver of disease due to loss of a section of chromosome 13 encoding for a wild-type BRCA2 copy. RNAseq performed on these archival tissues was successful in >40% of cases (26/64), but did not produce sufficient numbers for meaningful statistical analysis. Therefore, the response prediction (especially for the patient population described herein) may also include an analysis of DNA and/or RNA that identifies zygosity status (e.g., heterozygous, homozygous, loss of heterozygosity) for the pathogenic dinucleotide BRCA2 at Chr13 bases 32,914,102 (T->A) and 32,914,103 (C->G).

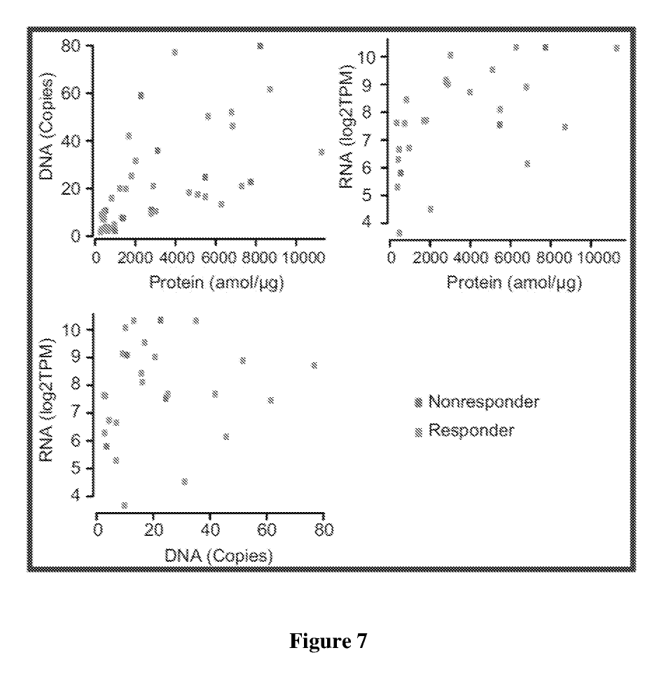

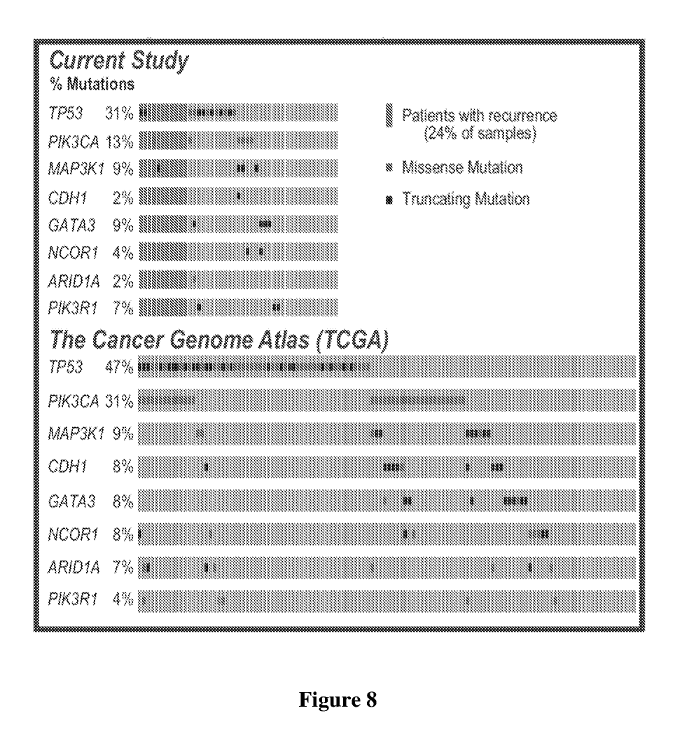

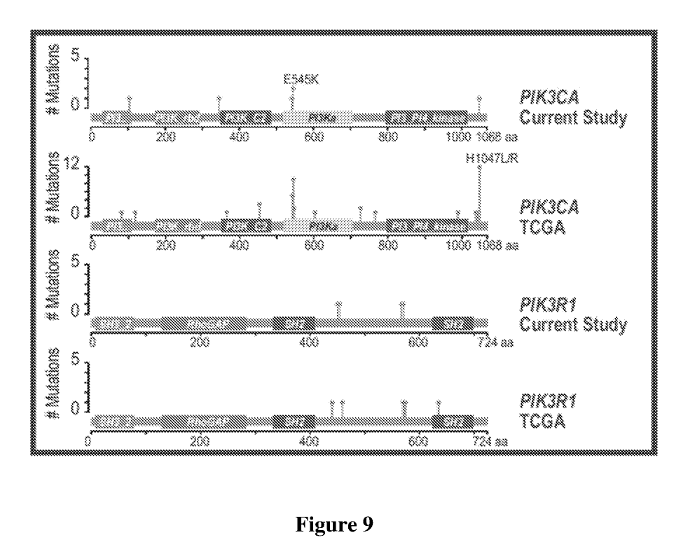

Tumor diversity was further evidenced by comparing DNA to protein for responders and non-responders, DNA to RNA (transcription) for responders and non-responders, and RNA to protein (translation) for responders and non-responders as is depicted in FIG. 7. Here, responders and non-responders were substantially randomly distributed in each of the plots, indicating no predictive pattern for HER2. When comparing the data of the present study against publicly available data from TCGA for HER2 positive patients as illustrated in FIG. 8, the rates of TP53 and PIK3CA mutations are different (Fisher's exact test, TP53 P=0.0794; PIK3CA P=0.0279), potentially due to enrichment of patients with metastatic disease. This is also evident from FIG. 9 where the mutational patterns between the present study and the TCGA data are also somewhat divergent for PIK3ACA and PIK3R1.

Consequently, based on these and other data (not shown), the inventors contemplate a method of predicting post-treatment relapse in a patient that is treated for a HER2-positive breast cancer, wherein the treatment comprises administration of an anti-HER2 antibody and at least an anthracycline and a taxane. Most typically, a breast cancer sample from the patient (e.g., fresh biopsy, frozen sample, FFPE sample, etc.), and the sample is then subjected to one or more omics or gene/protein specific tests to determine in the breast cancer sample the presence and/or quantity of TLE3, XRCC1, RRM1, and/or MGMT. In addition, HER2 is also specifically contemplated as a marker. The so determined presence and/or quantity is then used to predict the likelihood of post-treatment relapse in the patient.

With respect to marker determination, it is typically preferred (but not necessary) that the determination is not only qualitative, but also quantitative. For example, quantitative marker determination may be performed by determination of the copy number of the gene(s) that encodes the marker(s), and/or by determination of the absolute or relative number of transcripts (e.g., TPM, transcripts per million) of the gene(s) that encodes the marker(s), and/or by determination of protein quantities and/or activity. For example, contemplated HER2 protein quantification can be performed using various immunohistochemical (e.g., FISH) or immunological (e.g., ELISA) methods as described elsewhere (Breast Cancer Res 2015; 17(1): 41), or using mass spectroscopic methods such as SRM-MS or MRM-MS. Of course, it should be appreciated that such methods also include the quantification of activated proteins (e.g., phosphorylated forms). On the other hand, protein activity may also be determined using quantitative activity assays that are well known in the art (e.g., TLE3 assay as described in J Exp Clin Cancer Res 2016 Sep. 27; 35(1):152; XRCC1 as described in Methods 2016 Oct. 1; 108:99-110; RRM1 as described in PLoS One 2013; 8(7): e70191)

With respect to samples suitable for analysis it is contemplated that all samples are deemed appropriate for use herein and especially include fresh biopsy samples, frozen biopsy samples, processed biopsy samples (FFPE, formalin fixed, etc.), and liquid biopsy samples including exosomes, circulating bound and non-bound nucleic acids. Moreover, it should be appreciated that in some aspects the sample will also include a matched normal sample (i.e., a healthy or non-tumor sample from the same patient) to so allow for differential analysis without need for external reference information. In addition, it should be noted that suitable samples may also be processed to enrich for one or more specific analytes. For example, the sample processing may include nucleic acid or protein enrichment and/or purification, and suitable samples will therefore also include isolated nucleic acids (DNA and/or RNA) or isolated or otherwise tagged proteins/peptides. In still further aspects of the inventive subject matter, the sample may also have been previously processed, for example, to obtain sequence information. Therefore, suitable nucleic acid samples may also include sequence data in various file formats representing whole genome sequence data, whole exome sequence data, and/or RNAseq sequence data. Thus, the information may include raw sequences, aligned sequences, identification of base and/or structural changes, copy number information, and zygosity information. Likewise, protein information may also be present as predetermined quantitative and/or qualitative information (e.g., from FISH analysis, or mass spectroscopic analyses, etc). Consequently, it should be appreciated that the type of relevant omics analyses will vary considerably and suitable omics analyses include genomics analyses (DNA and/or RNA based analyses), transcriptomics analyses, proteomics analyses, and even microbiome analyses.

Moreover, it is noted that where specific markers are already identified, specific tests for selected markers may be designed or performed without further need for omics tests. For example, presence and/or quantity of TLE3, XRCC1, RRM1, and/or MGMT can be readily determined using conventional methods well known in the art. For example, suitable methods for qualitative and quantitative DNA detection include solid phase hybridization (e.g., microarray or bead based), LCR, qPCR, etc., while suitable methods for qualitative and quantitative RNA detection include quantitative rtPCR, RNAseq, etc. Likewise, suitable methods for qualitative and quantitative protein detection include mass spectroscopic analyses (and especially SRM-MS and other types of reaction monitoring MS), antibody-based detection, and ligand-based detection.

Depending on the particular type of test, it should be appreciated that the so detected analyte may be qualitatively (e.g., present or absent) or quantitatively (e.g., using absolute values or values normalized against, for example, matched normal) confirmed. For example one or more tests confirming presence and/or quantity of TLE3, XRCC1, RRM1, and/or MGMT, where the presence and/or quantity of TLE3, XRCC1, RRM1, and/or MGMT is indicative of likely treatment responder status (e.g., having low likelihood of post-treatment relapse in the patient). Such tests may especially include quantitative results where a correlation between the marker and the strength of the responder status exists (e.g., as is the case with TLE3).

Upon determination of the test result and likelihood of post-treatment relapse in the patient, the patient chart may be updated accordingly, and/or a treatment recommendation may be made to the medical professional or patient in care of the professional. Moreover, it should be noted that the test can be performed prior to treatment, during treatment, or after treatment, and that the timing and outcome of the test may determine the course of further action. For patients that were determined likely responders, treatment options for the HER2 cancer will therefore include three administration cycles of FEC (5-fluorouracil (5FU), epirubicin, and cyclophosphamide) and three administration cycles of docetaxel or docetaxel plus gemcitabine, or adjuvant chemotherapy with an anthracycline and a taxane. In either event, administration of an anti-HER2 antibody over a suitable period of time (e.g., 12 months or otherwise as indicated by the treating physician) will accompany the drug therapy.

### Examples

Matched tumor-normal samples (FFPE tumors and whole blood) underwent WGS; provenance testing was done to ensure specimen purity. WGS data were processed using Contraster. RNAseq of matched tumor-normal samples was performed to confirm the presence of gene mutations and was used to identify mutational and transcript abundance. Proteomics analysis was performed using a quantitative, multiplexed, selected reaction monitoring-mass spectrometry (SRM-MS) assay comprising a panel of 52 proteins. Tumor areas from FFPE tissue sections were laser microdissected, solubilized, and enzymatically digested. Absolute quantitation of proteins was accomplished through the simultaneous detection of endogenous targets and identical, synthetic, labeled heavy peptides; protein levels were normalized to total protein extracted from each sample.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

All methods described herein can be performed in any suitable order unless otherwise indicated herein or otherwise clearly contradicted by context. The use of any and all examples, or exemplary language (e.g., ‘such as’) provided with respect to certain embodiments herein is intended merely to better illuminate the invention and does not pose a limitation on the scope of the invention otherwise claimed. No language in the specification should be construed as indicating any non-claimed element essential to the practice of the invention.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

What is claimed is:

1. A method of predicting post-treatment relapse in a patient treated for a HER2-positive breast cancer, wherein the treatment comprises administration of an anti-HER2 antibody and at least an anthracycline and a taxane, comprising:
obtaining a breast cancer sample from the patient and determining in the breast cancer sample at least one of a presence and quantity of a marker selected from the group consisting of TLE3, XRCC1, RRM1, and MGMT; and
using the at least one of the presence and quantity of the marker to predict a likelihood of post-treatment relapse in the patient.

2. The method of claim 1 wherein the treatment comprises three administration cycles of FEC (5-fluorouracil (5FU), epirubicin, and cyclophosphamide) and three administration cycles of docetaxel or docetaxel plus gemcitabine.

3. The method of claim 1 wherein the treatment comprises an adjuvant chemotherapy with an anthracycline and a taxane.

4. The method of any of claim 1 or claim 2 wherein the administration of the anti-HER2 antibody is performed over 12 months.

5. The method of claim 1 wherein the step of determining the at least one of the presence and quantity of the marker is performed using at least one of DNA omics analysis, RNA omics analysis, and proteomics analysis.

6. The method of claim 1 wherein the step of determining the at least one of the presence and quantity of the marker is performed using at least two of DNA omics analysis, RNA omics analysis, and proteomics analysis.

7. The method of claim 1 wherein the step of determining the at least one of the presence and quantity of the marker is performed using DNA omics analysis, RNA omics analysis, and proteomics analysis.

8. The method of claim 1 wherein the step of determining the at least one of the presence and quantity of the marker includes at least one of determination of gene copy number, gene expression level, and protein level.

9. The method of claim 1 wherein the step of predicting likelihood of post-treatment relapse in the patient is independent of a size of a primary tumor, a lymph node status, a grade, and a hormone receptor status.

10. The method of claim 1 wherein the step of predicting likelihood of post-treatment relapse in the patient is not correlated with a HER2 quantity in the breast cancer sample.

11. The method of claim 1 wherein presence, increased copy number, or increased presence of the marker is predictive of lower likelihood of post-treatment relapse.

12. Use presence and/or quantity of at least one of TLE3, XRCC1, RRM1, and MGMT in the prediction of a treatment outcome of a HER2-positive breast cancer, wherein treatment comprises administration of an anti-HER2 antibody and at least an anthracycline and a taxane.

13. The use of claim 12 wherein the treatment comprises three administration cycles of FEC (5-fluorouracil (5FU), epirubicin, and cyclophosphamide) and three administration cycles of docetaxel or docetaxel plus gemcitabine.

14. The use of claim 12 wherein the treatment comprises an adjuvant chemotherapy with an anthracycline and a taxane.

15. The use of any of claim 13 or claim 14 wherein the administration of the anti-HER2 antibody is performed over 12 months.

16. The use of claim 12 wherein the presence and/or quantity are determined using at least one of DNA omics analysis, RNA omics analysis, and proteomics analysis.

17. The use of claim 12 wherein the presence and/or quantity are determined using at least two of DNA omics analysis, RNA omics analysis, and proteomics analysis.

18. The use of claim 12 wherein the presence and/or quantity are determined using DNA omics analysis, RNA omics analysis, and proteomics analysis.

19. The use of claim 12 wherein the presence and/or quantity are determined by measuring at least one of a gene copy number, a gene expression level, and a protein level.

20. The use of claim 12 wherein the prediction of a treatment outcome is independent of a size of a primary tumor, a lymph node status, a grade, and a hormone receptor status.
